(19) United States
(12) Patent Application Publication
(10) Pub. No.: US 2023/0172762 A1
(43) Pub. Date: Jun. 8, 2023

(54) SWAB FOR TAKING BIOLOGICAL SAMPLES AND METHOD FOR MAKING SUCH A SWAB

(71) Applicant: VACUTEST KIMA S.R.L., Arzergrande (Padova) (IT)

(72) Inventors: Renzo CHIARIN, Arzergrande (Padova) (IT); Ulisse CHIARIN, Arzergrande (Padova) (IT)

(21) Appl. No.: 17/924,098

(22) PCT Filed: May 18, 2021
(86) PCT No.: PCT/IB2021/054265

§ 371 Date: Nov. 8, 2022

(30) Foreign Application Priority Data
May 20, 2020 (IT) 102020000011683

(51) Int. Cl. A61F 13/38; A61B 10/00

(52) U.S. Cl. CPC A61F 13/385; A61B 10/0045

(57) ABSTRACT

A swab for taking biological samples which has a support rod having an end portion coated with a layer of fibres is provided. Each of the fibres extends in length orthogonally to a surface of the support rod with a respective free end portion. The layer of fibres has hydrophilic properties to allow absorption of biological samples. Each of the fibres is mechanically anchored at a bottom portion thereof to a textile substrate fixed to a surface of the end portion of the support rod.

### DESCRIPTION

### Field of Application

The present invention relates to a swab for taking biological samples, and to a method for making such a swab.

### Background Art

In the field of clinical and diagnostic analyses it is known to take biological samples of organic material from patients, in particular from the oral-nasal, ocular, rectal, urethral, or vaginal cavities, by means of sampling devices referred to as swabs.

A swab essentially consists of a support rod which, at a free end or tip portion thereof, is coated with a fibrous layer, consisting of synthetic fibres (e.g., rayon, polyester, polyamide) or natural fibres (e.g., cotton). The layer of fibres is made so to have hydrophilic properties so as to allow a rapid absorption of an amount of sample to be taken and tested. Generally, the fibres forming the fibrous layer are fixed to the rod by a layer of adhesive.

There are two main types of swabs on the market, which differ from each other in the manner in which the fibrous layer is made: cotton swabs and flocked swabs.

Cotton swabs are made by winding a wad of fibres around a tip portion of the support rod, on which a layer of adhesive has been previously applied.

Cotton swabs ensure a good liquid sample absorption capacity and are generally used for seeding on culture mediums to check for bacterial growth. Very often, after taking the sample, the absorbent part of the swab is stored inside a test-tube containing a gelatinous substance, referred to as transport medium, which has the task of preserving the taken sample until the analytical step. An example of such a type of device is described in European patent EP643131B1.

At the time of analysis, the release of the sample from a cotton swab occurs simply by grasping the swab rod and gently swiping the tip with the fibrous layer impregnated with liquid, for example, on a Petri dish with culture medium. In practice, the sample is spread on the dish with an operation referred to as swabbing.

Although the swabbing operation is repeated and accurate, it does not, however, allow the entire absorbed sample volume to be released since the part of the sample which has penetrated inside the volume of the wad, towards the tip, during such an operation is unable to be squeezed towards the surface and then released from the swab.

Because of this limit, on average, only 40% of the liquid sample taken can be recovered from a cotton swab for analysis. This leads to a reduction in the sensitivity of the analysis and an increase in false negatives.

This limit has been accepted for a long time also due to the absence of valid alternatives to cotton swabs.

In recent years, the market has begun to prefer liquid-type transport mediums in which the absorbent part of the swab is immersed, releasing a fraction of the sample taken. The thus contaminated liquid can be used directly for seeding or other investigations. This trend has intensified with the development of machines which perform complex operations such as the automatic seeding of liquid medium samples.

However, due to the low capacity of releasing the aforementioned sample, cotton swabs are absolutely not suitable for this type of use.

The need to obtain a greater release of the sample in the liquid medium has been satisfied with the advent of flocked swabs.

Flocked swabs are made by applying fibres by means of flocking to the tip portion of the swab support rod, as described in patent EP 1608268 B2.

The flocking process has been known for a long time in various fields: textiles, furniture, automotive, cosmetics.

Flocking consists in projecting natural or artificial fibres of small dimensions against a surface coated with adhesive.

Generally, the energy to orient and accelerate the fibres, so as to make them penetrate the adhesive layer, is provided by an electrostatic field applied between the fibre dispenser and the surface to be flocked. Alternatively, the distribution and positioning of the fibres can be obtained mechanically by means of appropriate vibrators.

The fibres used for the flocking process can have lengths ranging from a few tenths of a millimetre to a few millimetres and very low counts. They can be obtained with a physical-chemical process from polyamide, rayon, viscose, cotton fibres in an infinite variety of colours and sizes.

The fibres deposited with the electrostatic flocking technique tend to arrange themselves in an orthogonal manner with respect to the gluing surface; this arrangement of the fibres favours the absorption of the liquid sample and the subsequent release thereof into the transport medium.

As highlighted in patent EP 1608268 B2, a flocked swab ensures a release of up to 90% of the absorbed sample, much higher than that of a cotton swab, which is about 40%.

However, flocking is an onerous process which requires long production times and complex plants. In particular, after applying the fibres it is necessary to wait for the glue to dry and then clean the swab of any residual fibres which have not adhered to the adhesive layer, or of particles which have detached from the adhesive layer. The cleaning is carried out by brushing, vacuuming, or blowing, for example. This further complicates the production process.

There is therefore a need in the field to make swabs with fibres arranged orthogonally to the surface of the support rod without however using the flocking technique.

To date, such a need is still unmet.

### Presentation of the Invention

Therefore, it is the object of the present invention to eliminate or at least mitigate the drawbacks of the prior art mentioned above, by providing a swab for taking biological samples which has a layer of fibres arranged orthogonally to the surface of the support rod and which can be made in an operatively simpler manner than a flocked swab.

It is a further object of the present invention to provide a swab for taking biological samples which has a biological sample absorption and release capacity which is comparable to that of a flocked swab.

### BRIEF DESCRIPTION OF THE DRAWINGS

The technical features of the invention according to the aforesaid objects may be clearly found in the contents of the claims hereinbelow and the advantages thereof will become more apparent from the following detailed description, given with reference to the accompanying drawings which show one or more embodiments merely given by way of non-limiting example, in which:

FIGS. 1a, b, c, d, e show some examples of swabs according to the invention;

FIG. 2 shows some examples of support rods which can be used to make a swab according to the invention;

FIG. 3 shows a diagrammatic plan view of a portion of a textile substrate with fibres which can be used to make a swab according to the invention;

FIG. 3a shows a diagrammatic side view of the textile substrate portion shown in FIG. 3;

FIG. 4 shows in sequence the modes of winding a textile substrate with fibres on a support rod in accordance with a first embodiment of the method for making swabs according to the invention;

FIG. 5 shows in sequence the modes of winding a textile substrate with fibres on a support rod in accordance with a second embodiment of the method for making swabs according to the invention;

FIG. 6 shows a diagrammatic sectional view of a swab according to the invention;

FIGS. 7 and 8 show two details of FIG. 6 at different enlargements; and

FIGS. 9 and 10 show in a diagrammatic and simplified manner the production steps of a velvet ribbon;

FIG. 11 shows Table A, in which the data related to comparative tests between swabs according to the invention and flocked swabs are collected, carried out having a sample volume available for absorption equal to 50 µL;

FIG. 12 shows Table B, in which the data related to comparative tests between swabs according to the invention and flocked swabs are collected, carried out having a sample volume available for absorption equal to 100 µL; and

FIG. 13 shows Table C, in which the data related to comparative tests between swabs according to the invention and flocked swabs are collected, carried out having a sample volume available for absorption equal to 200 µL.

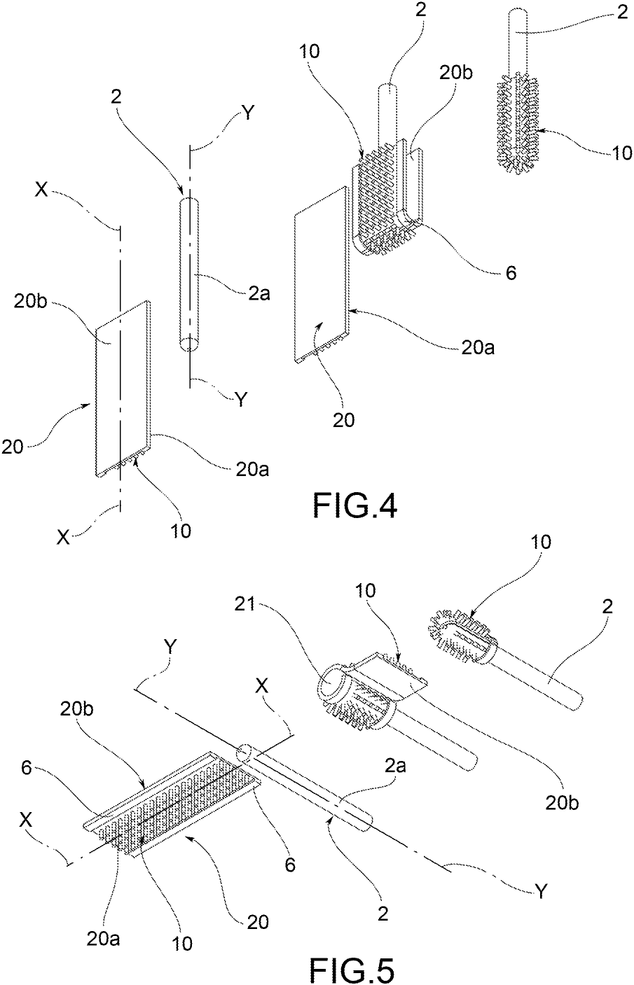

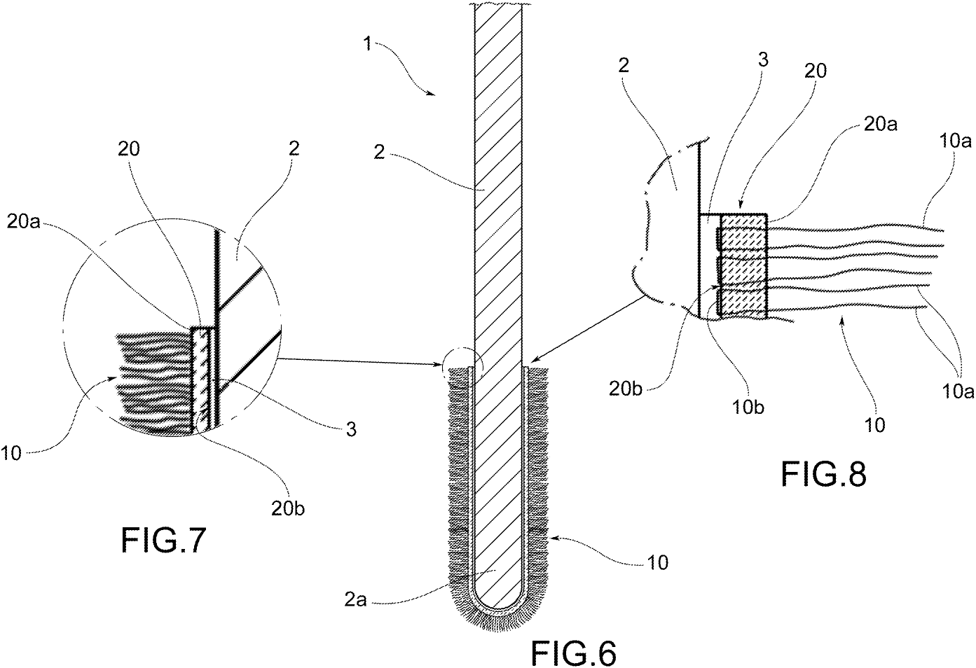

The images used in FIGS. 4, 5, 9 and 10 are intentionally out of scale, with details depicted not in proportion, to highlight details otherwise not graphically depictable.

### DETAILED DESCRIPTION

The present invention relates to a swab for taking biological samples, intended in particular for taking samples from the oral-nasal, ocular, rectal, urethral, or vaginal cavities of a patient.

With reference to the accompanying drawings, reference numeral 1 indicates as a whole a swab for taking biological samples according to the invention.

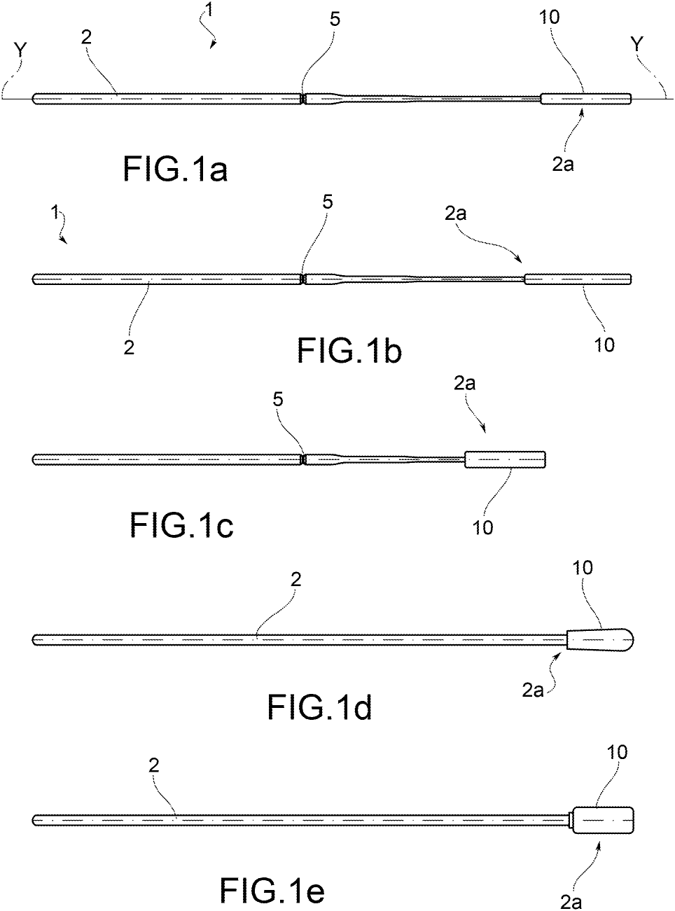

In accordance with a general embodiment of the invention, the swab 1 for taking biological samples comprises a support rod 2 having an end portion 2a coated with a layer of fibres 10. Some examples of swabs 1 are diagrammatically depicted in FIGS. 1a, b, c, d, e.

The support rod 2 can be of any length and shape according to the specific requirements which the swab must fulfil.

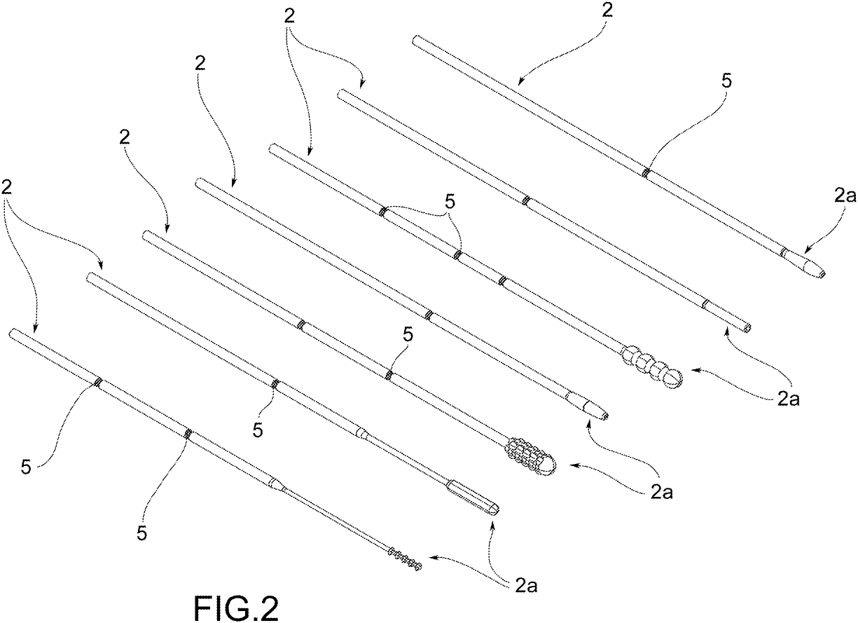

In particular, as depicted in FIG. 2, the end portion 2a intended to be coated can be cylindrical, ogival in shape or have protruding appendages defining localized enlargements. In particular, the support rod 2 can be provided with one or more circumferential bottlenecks 5, included as breaking points of the rod 2, so as to separate the end portion coated with the layer of fibres 10 from the rest of the rod 2.

The rod 2 can be made of any suitable material, which gives an adequate rigidity to the rod itself to allow the operation thereof during use. Preferably, the rod 2 is made of plastic material, such as polypropylene, polyester, polyethylene, polyamide, shockproof polystyrene, etc.

Each of the fibres 10 of such a layer extends in length substantially orthogonally to the surface of the support rod 2 with a corresponding free end portion 10a.

The aforesaid layer of fibres 10 has hydrophilic properties to allow the absorption of biological samples. Preferably, the hydrophilic properties of the layer of fibres 10 result from the arrangement of the fibres themselves inside the layer and from the phenomena of capillarity induced by the mass of fibres.

According to the invention, each of the aforementioned fibres 10 is mechanically anchored at the bottom portion 10b thereof to a textile substrate 20. In turn, the textile substrate 20 is fixed to a surface of the end portion 2a of the rod 2.

By virtue of the invention, and unlike traditional flocked swabs, the fibres are not deposited on the support rod directly by flocking, but by means of a textile substrate. Thereby, the formation of the layer of fibres can be carried out separately from the step of fixing such a layer to the support rod. This significantly simplifies the manufacturing process of the swab 1, completely freeing it from the flocking technique.

As already mentioned, the function of absorbing biological samples is performed by the layer of fibres 10, mainly due to capillarity phenomena between the fibres.

The amount of biological sample which can be collected from a swab 1 depends on the features of the layer of fibres 10 (fibre count, length, surface distribution density) and on the surface extension of the layer of fibres 10.

Advantageously, as a function of the features of the substrate 20, the substrate 20 itself can also partially contribute to the absorption function of the biological samples, in addition to the layer of fibres. Differently, the layer of adhesive to which the fibres are directly fixed in flocked swabs cannot contribute to the absorption of biological samples.

Preferably, each of the aforementioned fibres 10 is wedged in the weave of the textile substrate so as to be mechanically retained thereby.

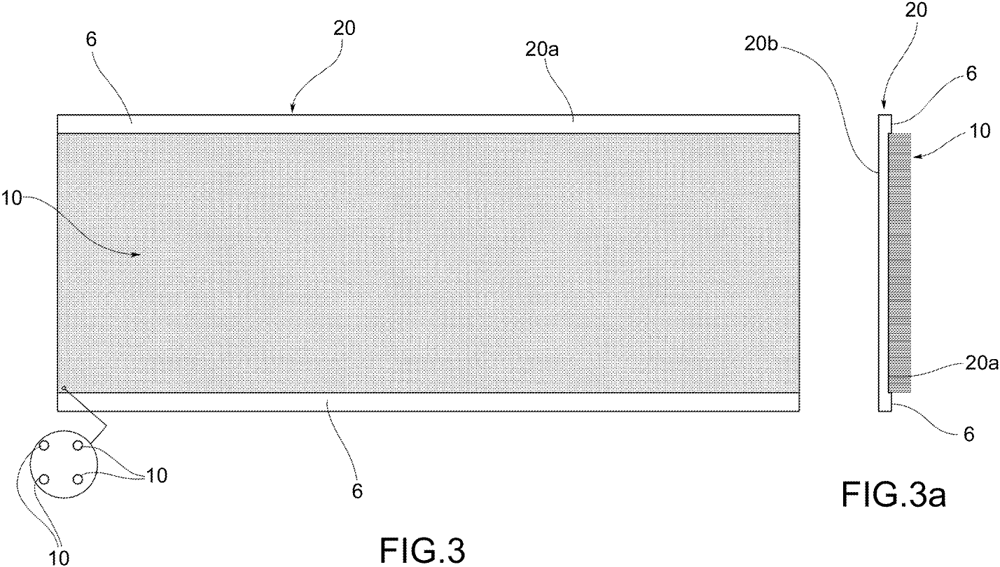

As shown in particular in FIG. 3a, the following can be identified on the textile substrate 20:
an outer face 20a, from which the fibres 10 extend and which faces the outside of the swab 1; and
an inner face 20b, which is opposite the outer face and which adheres to the surface of the end portion 2a of the rod 2.

In particular, as diagrammatically shown in FIG. 8, each of the aforesaid fibres 10 crosses the textile substrate 20 so that:
the respective free end portion 10a extends orthogonally from the aforesaid outer face 20a of the textile substrate 20, and
the respective bottom portion 10b extends from the inner face 20b of the textile substrate 20 and is arranged between the inner face 20b and the surface of the end portion 2a of the rod 2.

Preferably, as diagrammatically shown in FIGS. 8, 9 and 10, the aforesaid fibres 10 are joined together in pairs at the respective bottom portions 10b to form a single body.

More in detail, each pair of “joined” fibres is defined by a single fibre piece passing through the substrate 20 in two different points. Thereby, the two ends of such a piece, protruding from the outer face 20a of the substrate 20 at two different points, form the free end portions 10a of two fibres 10, while the portion of the piece which is located below the substrate, adjacent to the inner face 20b of the latter, forms the bottom portions 10b of such two fibres 10.

In particular, such an arrangement of the fibres can be easily obtained by means of a simple process of sewing the fibres onto the substrate.

Advantageously, the textile substrate 20 and the fibres 10 are associated with the aforesaid end portion 2a of the rod 2 as a pre-assembled body.

In accordance with a wholly preferred embodiment of the invention, the aforesaid pre-assembled body consists of a velvet ribbon.

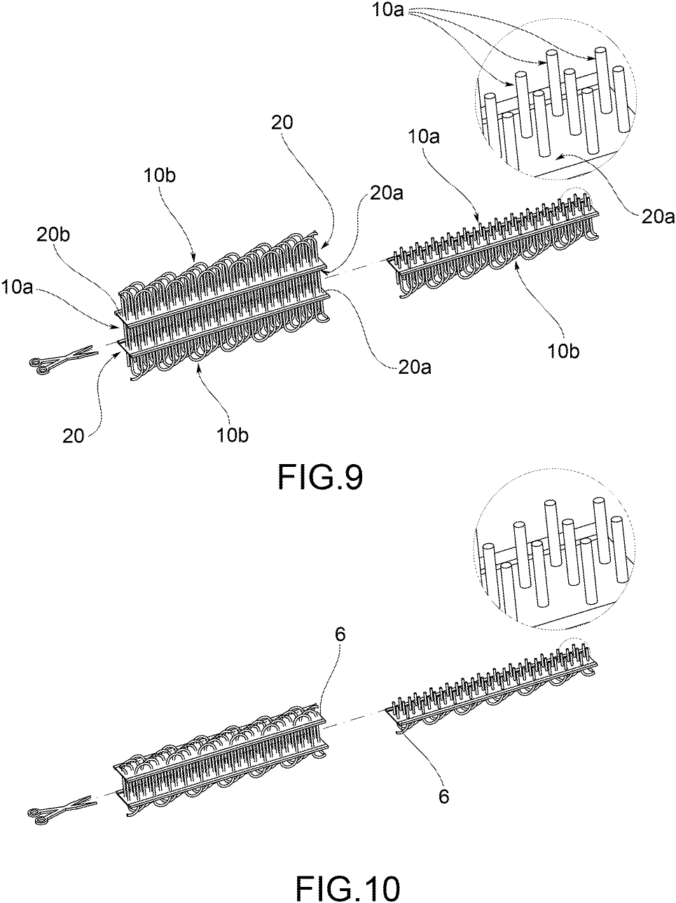

The production process of a velvet ribbon is well known per se and is diagrammatically shown in FIGS. 9 and 10. More in detail, two textile ribbons, preferably edged in a longitudinal direction by selvedges 6, are tightly sewn together, until the surface between the two edges of the ribbon is filled with stitches. The two ribbons are then separated from each other by cutting the sewing threads at the gap existing therebetween. The cut sewing threads separate, creating a layer of free hairs (fibres) on one face of each ribbon, which are anchored to the ribbon (textile substrate) at the bottom thereof. The density of the free hair layer depends on the density of the stitches.

Preferably, the fibres 10 extend from the textile substrate 20 outwards with substantially the same length. In other words, the layer of fibres 10 has a substantially uniform height.

Preferably, such a length is between 0.5 mm and 3 mm, and even more preferably between 1.0 mm and 1.30 mm.

Preferably, the aforementioned fibres 10 are uniformly distributed on the textile substrate 20. Even more preferably, the aforementioned fibres 10 are uniformly distributed on the textile substrate 20 with a surface density between 80 and 130 fibres/mm2.

Preferably, the aforesaid fibres 10 have a count between 60 dtex and 90 dtex, and even more preferably between 70 dtex and 80 dtex.

The use of fibres within the aforesaid count ranges allows to increase the mechanical strength of the fibres themselves, reducing the risk of accidental breakage of the fibres themselves and consequent dispersion thereof during the operations of taking biological samples.

Furthermore, the use of fibres within the aforesaid count ranges allows to reduce the incidence of intertwining fibres compared to the use of fibres with lower counts.

It has been verified that the use of fibres within the aforesaid count ranges does not negatively affect the absorption capacity of the layer of fibres.

The fibres 10 can be:
in synthetic material, preferably selected from the group consisting of rayon, polyester, polyamide, polypropylene, polyethylene, or
in natural material, preferably selected from the group consisting of cotton and silk, or
blends thereof.

In accordance with a preferred embodiment of the invention, the fibres 10 are made of polyamide, preferably nylon 6 or nylon 6,6.

Preferably, the aforesaid textile substrate 20 is fixed directly to the surface of the end portion 2a of the rod 2.

Even more preferably, the aforesaid textile substrate 20 is glued to the surface of the end portion 2a of the rod 2. As diagrammatically shown in FIG. 7, a layer of adhesive 3 can form between the textile substrate 20 and the surface of the rod 2, which can partially penetrate the textile substrate 20. Preferably, however, the adhesive does not go beyond the textile substrate 20 so as not to invade the layer of fibres 10 extending from the outer face 20a of the substrate 20 and which come into direct contact with the patient’s body in use.

Advantageously, as diagrammatically shown in FIG. 8, the adhesive layer 3 can, however, come into contact with the bottom portions 10b of the fibres. This further contributes to stabilizing the fibres 10 on the swab 1, reducing the risk of accidental detachment of the fibres themselves.

Alternatively, other modes of fixing the substrate 20 to the rod 2 can be included, for example by heat or ultrasound, as a function of the material with which the substrate itself is made. However, gluing is the preferred method.

The aforesaid textile substrate 20 can be made with threads:
in synthetic material, preferably selected from the group consisting of rayon, polyester, polyamide, polypropylene, polyethylene, or
in natural material, preferably selected from the group consisting of cotton and silk, or
blends thereof.

In accordance with a preferred embodiment of the invention, said textile substrate 20 consists of polyamide threads, preferably nylon 6 or nylon 6,6.

Preferably, the textile substrate 20 is made of the same material as the fibres 10, although different materials can also be used.

Preferably, the textile substrate 20 consists of threads having a count between 30 dtex and 120 dtex. The warp and weft threads can have the same count. However, it is possible to differentiate the count of the weft threads with respect to the warp threads. For example, it is possible to make a textile substrate in which the weft threads have a count of 33 dtex, while the warp threads have a count of 110 dtex.

Preferably, the aforesaid textile substrate 20 has a weight between 20 g/m2 and 600 g/m2.

Advantageously, the weight of the substrate 20 is chosen as a function of the contribution in terms of absorption of the biological samples to be obtained from the substrate 20.

The present invention also relates to a kit for taking and transporting biological samples. Such a kit comprises a test-tube containing a culture medium (preferably liquid), and a swab 1 according to the invention, and in particular as described above.

The present invention also relates to a method for making a swab 1 for taking biological samples according to the invention, and in particular as described above.

In accordance with a form of general implementation of the invention, the method comprises the following operating steps:
a) providing a support rod 2 comprising a free end portion 2a;
b) providing a textile substrate 20 comprising a first face 20a from which a plurality of fibres 10 mechanically anchored to said textile substrate 2 extends orthogonally; and
c) covering the free end portion 2a of said rod 2 with the textile substrate 20.

The covering step c) is carried out by fixing the textile substrate 20 to the rod 2 at a second face 20b of the substrate 20, opposite said first face 20a, so that the fibres 10 extend orthogonally from the surface of the free end portion 2a of the rod 2 covered by the substrate, forming a layer of fibres 10 which extends all around the aforesaid free end portion 2a.

In accordance with a preferred embodiment of the method, the textile substrate 20 provided with fibres 10 consists of a velvet ribbon.

Preferably, the aforesaid textile substrate 20 is fixed to the rod 2 by gluing by application of an adhesive to said textile substrate 20 and/or to the rod 2. The substrate 20 is adhered to the portion of the rod to be coated by interposing a layer of glue/adhesive between the substrate and the rod.

As already mentioned, other modes of fixing the substrate 20 to the rod 2 can alternatively be included, for example by heat or ultrasound, as a function of the material with which the substrate itself is made. However, gluing is the preferred method.

Preferably, the method comprises a step d) of applying a glue on the second face 20b of said textile substrate 20. The application of the glue / adhesive is carried out before the covering step c).

The adhesive/glue can be applied on the rod 2, rather than on the textile substrate. However, it is preferable to apply the adhesive on the substrate rather than on the rod 2 as this is not only operatively simpler but ensures a better and more precise fixing of the substrate on the rod.

Advantageously, the aforesaid glue/adhesive is chosen with a sufficiently high viscosity as not to completely penetrate the textile substrate 20 and thereby not be absorbed by capillarity by the fibres 10 extending from the first face 20a of the substrate 20.

Preferably, the aforesaid textile substrate 20 is ribbon-shaped having a prevailing longitudinal direction of extension X, while the free end portion 2a of the rod 2 has a prevailing extension axis Y.

In accordance with a first embodiment of the method according to the invention, diagrammatically shown in FIG. 5, during the aforesaid covering step c) the textile substrate 20 (in the form of a ribbon) is wound along the aforesaid prevailing longitudinal direction of extension X on said free end portion 2a of the rod 2 around the prevailing extension axis Y until it covers all said free end portion 2a.

As a function of the height of the ribbon and the length of the free end portion 2a to be coated, the winding may require a single turn, or several turns, for example according to a spiral pattern.

Preferably, the substrate 20 is wound around said free end portion 2a so that a portion 21 of the substrate 20 itself protrudes beyond the free end of the rod 2. The portion 21 of substrate protruding beyond the free end of the rod 2 is partially cut. In particular, the selvedge of the edge is cut, and the dimensions of the protruding portion are adapted to the portion of the rod still to be covered. The residual protruding strips are then forced to close radially on the free end 2a to form a continuous surface also at the free end of the rod 2.

The winding mode described above (FIG. 5) is particularly suitable for coating portions of rods with larger diameters.

In accordance with a second embodiment of the method according to the invention, diagrammatically shown in FIG. 4, during the aforesaid covering step c) a portion of textile substrate 20 (in the form of a ribbon) is placed at the centre thereof on the end of the rod 2. The two symmetrical strips of the substrate are then folded on the rod 2 so as to form a cap which is then pressed so as to adhere to the rod itself.

Preferably, the excess ribbon (and in particular the selvedges of the edges) is cut along cutting lines parallel to the prevailing extension axis Y of the rod 2 and the remaining ribbon is made to adhere to the rod to obtain a continuous surface along said cutting lines.

The winding mode just described (FIG. 4) is particularly suitable for coating portions of rods with smaller diameters.

In general, the method according to the invention, and in particular the two specific modes of winding the textile substrate described above, can be automated and allow to make swabs in a simple manner, making them immediately available for packaging, unlike production by flocking which requires long drying times and subsequent cleaning of excess fibres.

Comparative tests were conducted between flocked swabs and swabs made according to the invention. It was found that in many cases, with the same surface covered with fibres, the swabs according to the invention allow the absorption of a greater amount of biological sample than that of the flocked swabs. Such a result can be explained by the contribution in terms of absorption given by the textile substrate present in the swabs according to the invention.

It has also been verified that in many cases, with the same amount of sample available for absorption, the swabs according to the invention allow a higher release of the sample, which can be estimated at about 10%-15% more.

### Comparative Tests

Comparative tests were conducted between a swab according to the invention and a flocked swab among the most widespread on the market.

The swab according to the invention had the following features: — surface coated with fibres equal to 130 mm2; — textile substrate with a weight of 170 g/m2; warp threads 110 dtex; weft threads 33 dtex; protruding fibres 78 dtex and length 1.2 mm (extension with respect to the substrate); substrate and fibres made of 100% polyamide (nylon 6). The swab according to the invention had a surface coated with fibres equivalent to that of the flocked swab.

The comparative test was carried out in two parts: in the first part the amount absorbed by the swabs was evaluated, while in the second part (immediately sequential to the first part) the amount released by the swabs was evaluated.

To evaluate the amount absorbed by the swabs, the following procedure was carried out. Deionized water was taken from a container using a pipette and deposited in a dish. Several tests were conducted by varying the amount of water available in the dish and then by varying the amount absorbed by the swabs. The use of deionized water (pure grade II water) allows the density to be dependent only on temperature (and minimally) and the absorption to not be linked to the absorbed liquid/substrate affinity. Once the drop was placed on the dish, the amount in grams deposited was measured on a thousandth scale (division 1 mg) . Then the swab under examination was taken and, for a time of 5 seconds, an attempt was made to absorb the maximum amount of liquid by turning the swab around the drop. Once the swab was extracted, the remaining amount was measured in grams. The amount absorbed by the swab is equal to the difference between the deposited amount and the remaining amount. Differential measurement is a measurement which also has the advantage of being free from any absolute measurement error which the scale could have.

The following procedure was carried out to evaluate the amount released by the swabs. A previously prepared piece of filter paper (50 x50 mm squares) was placed on the scale and the scale was tared. Taking the swab just soaked in ionized water, it was rotated for 5 seconds around the filter paper while applying light pressure, in order to transfer the maximum amount of liquid from the swab to the filter paper. The entire surface of the filter paper was used. Also in this case, by means of differential measurement, the amount of liquid released by the swab was thus determined.

In each analysis, for each sample, two values were obtained: absorbed amount and released amount. Data analyses were then carried out on these two sets of values.

The tests were conducted on three different volumes of water available on the dish: 50 µL, 100 µL and 200 µL. The volumes 50 µL and 100 µL were chosen because they correspond to the volumes of bacterial suspension which are normally inoculated in quality control tests. On the other hand, the volume 200 µL was chosen to highlight how the trend of the absorption and release cycle stabilizes at infinite volume.

As regards the time available for both absorption and release, the value of 5 seconds was chosen, a time certainly used in the general clinical use of swabs.

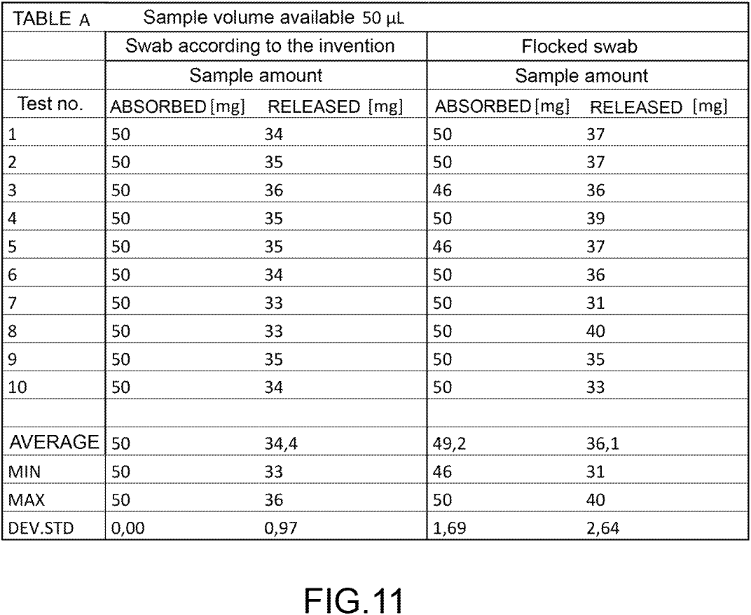

Table A, shown in FIG. 11, collects the data related to the comparative tests between the swab according to the invention and the flocked swab, carried out having a volume of water available on the dish equal to 50 µL.

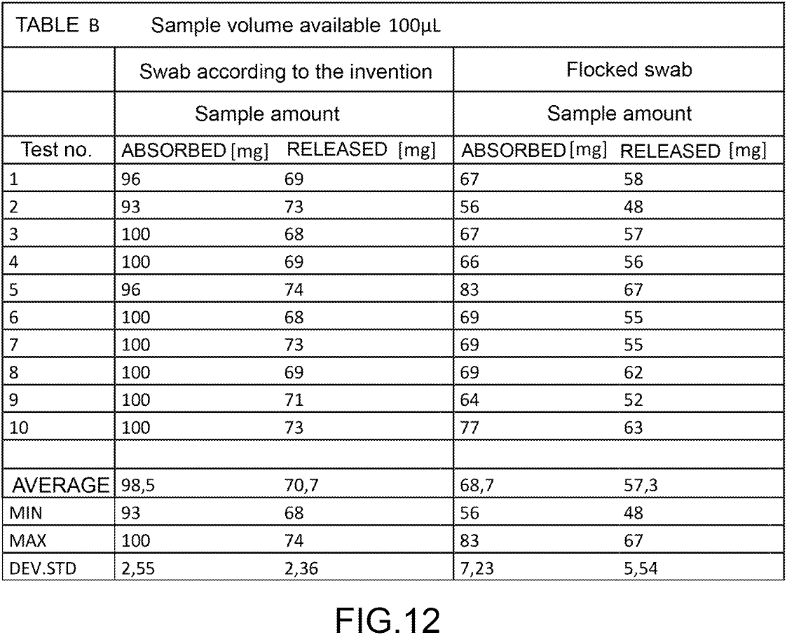

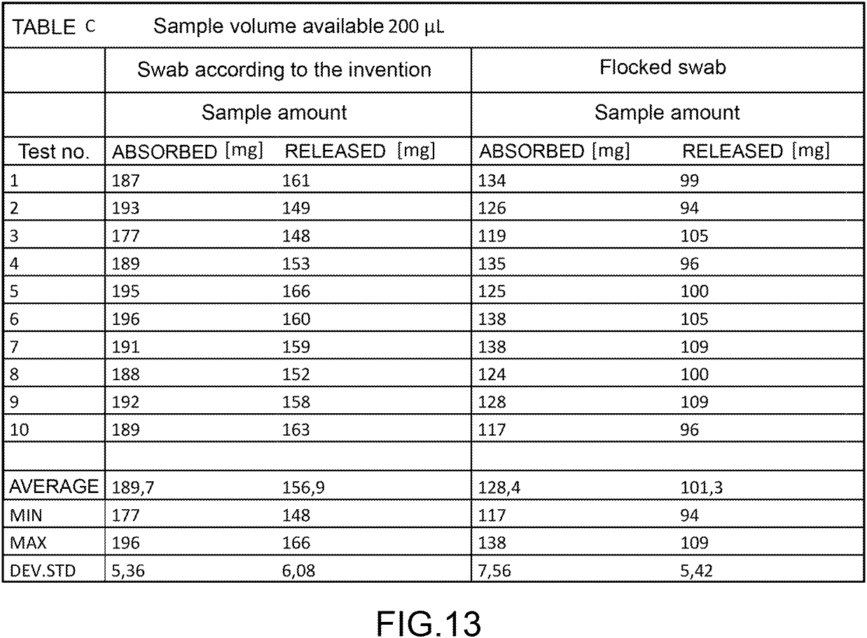

Table B, shown in FIG. 12, collects the data related to the comparative tests between the swab according to the invention and the flocked swab, carried out having a volume of water available on the dish equal to 100 µL.

Table C, shown in FIG. 13, collects the data related to the comparative tests between the swab according to the invention and the flocked swab, carried out having a volume of water available on the dish equal to 200 µL.

The comparative tests conducted highlight that the swab according to the invention has features and functionality comparable to the flocked swabs available on the market, which currently represent the standard in the microbiological field, especially as regards virological analyses.

In particular, the tests highlighted a greater absorption capacity in absolute terms of the swab according to the invention with respect to flocked swabs, both as regards samples of 100 µL and 200 µL, and as regards samples of 50 µL.

As regards the release, it can be noted that in the case of samples with a volume of 50 µL the releases in absolute terms are very similar between swabs according to the invention and flocked swabs, while in the cases of 100 µL and 200 µL the release in the swabs according to the invention is superior to the flocked swab.

Finally, the comparative tests demonstrated that the swab according to the invention, over a period of a few seconds (time corresponding to clinical use), absorbs a certain amount of sample, and is then able to release a large part of this absorbed amount.

It can therefore be concluded that a swab for taking biological samples according to the invention has at least a biological sample absorption and release capacity which is comparable to (if not even higher than) that of a flocked swab.

The invention allows to obtain several advantages which have been explained in the description.

The swab for taking biological samples according to the invention has a layer of fibres arranged orthogonally to the surface of the support rod and can be made in an operatively simpler manner than a flocked swab.

The swab for taking biological samples according to the invention has a biological sample absorption and release capacity which is comparable to that of a flocked swab.

Therefore, the invention thus conceived achieves the pre-set objects.

Obviously, in the practice thereof, it may also take other shapes and configurations than that disclosed above, without for this reason departing from the present scope of protection.

Moreover, all details may be replaced by technically equivalent elements, and any size, shape and material may be used according to the needs.

What is claimed is:

1. A swab for taking biological samples, the swab comprising a support rod comprising an end portion coated with a layer of fibres, each fibre of the layer of fibres extending in length orthogonally to a surface of the support rod with a respective free end portion, wherein said layer of fibres has hydrophilic properties to allow absorption of the biological samples, and wherein each of said fibres is mechanically anchored at a respective bottom portion to a textile substrate fixed to a surface of the end portion of said support rod.

2. The swab of claim 1, wherein each of said fibres crosses said textile substrate in such a way that the respective free end portion extends orthogonally from an outer face of said textile substrate , and the respective bottom portion extends from an inner face of said textile substrate, opposite said outer face, and is positioned between said inner face and the surface of the end portion of said support rod.

3. The swab of claim 2, wherein said fibres are joined together in pairs at respective bottom portions to form a single body.

4. The swab of claim 1, wherein said textile substrate and said fibres are joined to said end portion of said support rod as a pre-assembled body.

5. The swab of claim 4, wherein said pre-assembled body consists of a velvet ribbon.

6. The swab of claim 1, wherein said fibres extend from said textile substrate outwards substantially with a same length,.

7. The swab of claim 1, wherein said fibres are evenly distributed over said textile substrate.

8. The swab of claim 1, wherein said fibres have a count between 60 dtex and 90 dtex.

9. The swab of claim 1, wherein said fibres are in synthetic material or natural material.

10. The swab of claim 1, wherein said fibres are in polyamide.

11. The swab of claim 1, wherein said textile substrate is attached directly to the surface of the end portion of said support rod .

12. The swab of claim 11, wherein said textile substrate is glued to the surface of the end portion of said support rod.

13. The swab of claim 1, wherein said textile substrate is made of threads in synthetic material or threads in natural material.

14. The swab of claim 1, wherein said textile substrate consists of polyamide threads.

15. The swab of claim 1, wherein said textile substrate consists of threads having a count between 30 dtex and 120 dtex.

16. The swab of claim 1, wherein said textile substrate has a weight ranging between 20 g/m² and 600 g/m².

17. A kit for taking and transporting biological samples, said kit comprising a test-tube containing a culture medium, and a swab according to claim 1.

18. A method for making a swab for taking biological samples according to claim 1, said method comprising the following steps:
(a) providing a support rod comprising a free end portion;
(b) providing a textile substrate comprising an outer face from which a plurality of fibres mechanically anchored to said textile substrate extends orthogonally; and
(c) covering the free end portion of said support rod with said textile substrate by fixing said textile substrate to said support rod at an inner face opposite said outer face so that said fibres extend orthogonally from a surface of the free end portion of said support rod forming a layer of fibres extending around said free end portion.

19. The method of claim 18, wherein said textile substrate with fibres consists of a velvet ribbon.

20. The method of claim 18, wherein said textile substrate is fixed to said support rod by applying an adhesive to said textile substrate and/or to said support rod.

21. The method of claim 20, further comprising
(d) applying an adhesive to said inner face of said textile substrate, before said covering step.

22. The method of claim 20, wherein said adhesive is chosen with a sufficiently high viscosity not to penetrate completely the textile substrate and thereby not be absorbed by capillarity by the fibres extending from the outer face of said textile substrate .

23. The method of claim 18, wherein said textile substrate has a shape of a ribbon with a prevailing longitudinal direction of extension and the free end portion of said support rod has a prevailing extension axis, and wherein during said covering step said textile substrate is wound along said prevailing longitudinal direction of extension on said free end portion of said support rod around said prevailing extension axis until the textile substrate covers all said free end portion.

24. The method of claim 23, wherein said textile substrate is wound around said free end portion so that a portion of the textile substrate protrudes beyond the free end portion of the support rod, and wherein said portion of the textile substrate protruding beyond the free end portion of the support rod is partially cut and residual protruding strips are forced to close radially on the free end portion to form a continuous surface also at the free end portion of the support rod.

25. The method of claim 18, wherein said textile substrate has a shape of a ribbon with a prevailing longitudinal extension direction and the free end portion of said support rod has a prevailing extension axis, and wherein, during said covering step a portion of said ribbon is placed at its centre on the free end portion of the support rod and two symmetrical strips are bent on the support rod so as to form a cap which is then pressed to adhere to the support rod, .

26. The swab of claim 6, wherein said length ranges between 0.5 mm and 3 mm.

27. The swab of claim 6, wherein said length ranges between 1.0 mm and 1.30 mm.

28. The swab of claim 7, wherein the fibres are distributed over the textile substrate with a surface density ranging between 80 and 130 fibres/mm².

29. The swab of claim 9, wherein the synthetic material is selected from the group consisting of rayon, polyester, polyamide, polypropylene, and polyethylene, and the natural material is selected from the group consisting of cotton and silk, or blends thereof.

30. The swab of claim 13, wherein the synthetic material is selected from the group consisting of rayon, polyester, polyamide, polypropylene, and polyethylene, and the natural material is selected from the group consisting of cotton and silk, or blends thereof.

31. The method of claim 25, wherein excess ribbon is cut along cutting lines parallel to the prevailing extension axis of the support rod and remaining ribbon is made to adhere to the support rod to obtain a continuous surface along said cutting lines.
